(19) United States
(12) Patent Application Publication
(10) Pub. No.: US 2024/0382079 A1
(43) Pub. Date: Nov. 21, 2024

(54) METHOD FOR INSERTION INTO BILE DUCT AND ENDOSCOPIC TREATMENT TOOL

(71) Applicants: Tomoyasu HASEGAWA, Hachioji-shi (JP); Hirofumi ETO, Akiruno-shi (JP); Seiji IWASAKI, Hachioji-shi (JP); Takayoshi TSUCHIYA, Tokyo (JP); Mamoru TAKENAKA, Kobe-shi (JP); Eisuke IWASAKI, Matsudo-shi (JP)

(72) Inventors: Tomoyasu HASEGAWA, Hachioji-shi (JP); Hirofumi ETO, Akiruno-shi (JP); Seiji IWASAKI, Hachioji-shi (JP); Takayoshi TSUCHIYA, Tokyo (JP); Mamoru TAKENAKA, Kobe-shi (JP); Eisuke IWASAKI, Matsudo-shi (JP)

(21) Appl. No.: 18/320,554

(22) Filed: May 19, 2023

(51) Int. Cl. A61B 1/018; A61B 1/00

(52) U.S. Cl. CPC A61B 1/018; A61B 1/00042; A61B 1/00082; A61B 1/00087; A61B 1/00148; A61B 1/00154

(57) ABSTRACT

A method for insertion into a bile duct includes inserting a first device from a duodenum into an opening of a papilla, and advancing a second device within the bile duct while pulling the papilla with the first device.

### TECHNICAL FIELD

The present invention relates to a method for insertion into a bile duct and an endoscopic treatment tool.

### BACKGROUND

A procedure is performed in which a medical device such as a guide wire is inserted into a bile duct. Inserting a medical device into the bile duct is difficult because the bile duct has narrowed and tortuous portions. As described in U.S. Pat. No. 8,366,673, etc., medical devices that are easy to insert into bile ducts have been devised.

### SUMMARY

A method for insertion into a bile duct according to a first aspect of the present invention includes inserting a first device from a duodenum into an opening of a papilla, and advancing a second device within the bile duct while pulling the papilla with the first device.

An endoscopic treatment tool according to a second aspect of the present invention includes a sheath and a forceps jaw movable relative to the sheath, wherein an outer circumferential surface of the forceps jaw has at least one edge facing outward in a radial direction of the sheath, and the forceps jaw is connected to the sheath so that the edge can be moved in the radial direction outward relative to the outer peripheral surface of the sheath.

### BRIEF DESCRIPTION OF THE DRAWINGS

FIG. 1 is an overall view of an endoscopic treatment system according to a first embodiment.

FIG. 2 is an overall view showing a treatment tool of the endoscopic treatment system.

FIG. 3 is a perspective view of a distal end of the treatment tool.

FIG. 4 is a perspective view of the distal end of the treatment tool.

FIG. 5 is a diagram showing a bile duct to be treated.

FIG. 6 is a diagram showing a hooking step.

FIG. 7A is a diagram showing a second device insertion step.

FIG. 7B is a diagram showing the second device insertion step.

FIG. 8 is a diagram showing a guide wire placement step.

FIG. 9 is a diagram showing a stent placement step.

FIG. 10 is a diagram showing a modification of a guide wire lumen.

FIG. 11 is a diagram showing a modification of a forceps jaw.

FIG. 12 is a diagram showing another modification of the forceps jaw.

FIG. 13 is a diagram showing another modification of the forceps jaw.

FIG. 14 is a diagram showing another modification of the forceps jaw.

FIG. 15 is a diagram showing another modification of the forceps jaw.

FIG. 16 is a diagram showing the second device insertion step by the endoscopic treatment system according to the first embodiment.

### DETAILED DESCRIPTION

### First Embodiment

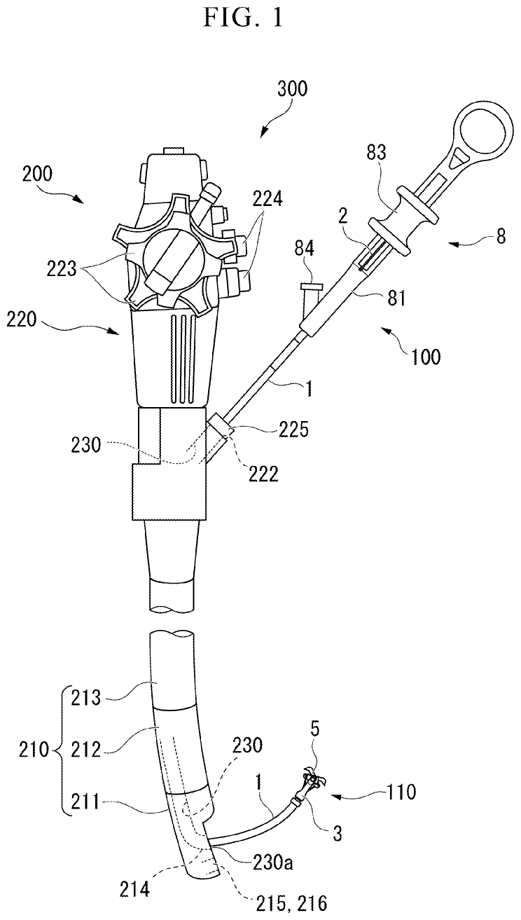

An endoscopic treatment system 300 including an endoscopic treatment tool 100 according to a first embodiment of the present invention will be described with reference to FIGS. 1 to 15.

### Endoscope Treatment System 300

FIG. 1 is an overall view of the endoscopic treatment system 300.

As shown in FIG. 1, the endoscopic treatment system 300 includes the endoscopic treatment tool 100 and an endoscope 200. The endoscopic treatment tool 100 is used by being inserted into the endoscope 200.

### Endoscope 200

The endoscope 200 is a known side-viewing flexible endoscope, and includes an elongated insertion portion 210 and an operating portion 220 provided at the proximal end of the insertion portion 210. The endoscope 200 may be a direct-viewing flexible endoscope.

The insertion portion 210 includes a rigid distal portion 211 provided at the distal end, a bending portion 212 provided on the proximal side of the rigid distal portion 211 that can be bent, and a flexible tube portion 213 provided on the proximal side of the bending portion 212. A light guide 215 and an imaging unit 216 having a CCD are provided on the side surface of the rigid distal portion 211 in a state where they are exposed to the outside.

A treatment tool channel 230 through which the endoscopic treatment tool 100 is inserted is formed in the insertion portion 210. A distal portion 230a of the treatment tool channel 230 is open on the side surface of the rigid distal portion 211. A proximal end of the treatment tool channel 230 extends to the operating portion 220.

A raising base 214 is provided on the rigid distal portion 211 of the treatment tool channel 230. A proximal portion of the raising base 214 is rotatably supported by the rigid distal portion 211. A raising base operating wire (not shown) fixed to the distal end of the raising base 214 extends through the insertion portion 210 toward the proximal end.

The bending portion 212 is configured to be freely curved in the vertical and horizontal directions. The distal end of the operating wire is fixed to the distal side of the bending portion 212. The operating wire extends through the insertion portion 210 to the operating portion 220.

A knob 223 for operating the operating wire, a switch 224 for operating the imaging unit 216, etc. are provided on the proximal side of the operating portion 220. The user can bend the bending portion 212 in a desired direction by operating the knob 223.

A forceps port 222 that communicates with the treatment tool channel 230 is provided on the distal end side of the operating portion 220. The user can insert the endoscopic treatment tool 100 through the forceps port 222. A forceps plug 225 is attached to the forceps port 222 to prevent leakage of body fluids.

### Endoscope Treatment Tool 100

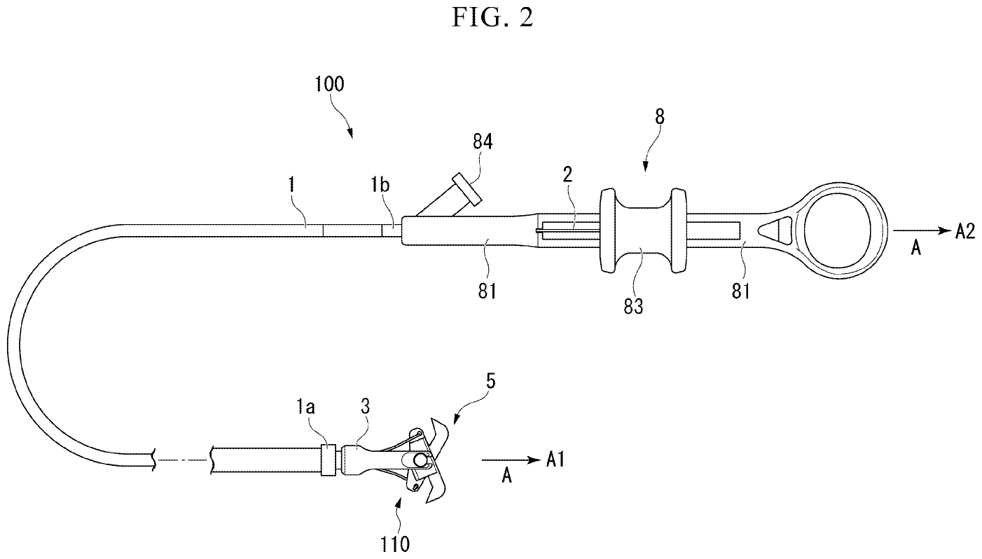

FIG. 2 is an overall view showing the endoscopic treatment tool 100.

The endoscopic treatment tool 100 (also referred to as the treatment tool 100) includes a sheath 1, an operating wire 2, a support member 3, a guide wire tube 4, a forceps jaw (first device) 5, and an operating portion 8. In the following description, in the longitudinal direction A of the treatment tool 100, the side inserted into the patient's body will be referred to as a “distal side A1”, and the side of the operating portion 8 will be referred to as a “proximal side A2”.

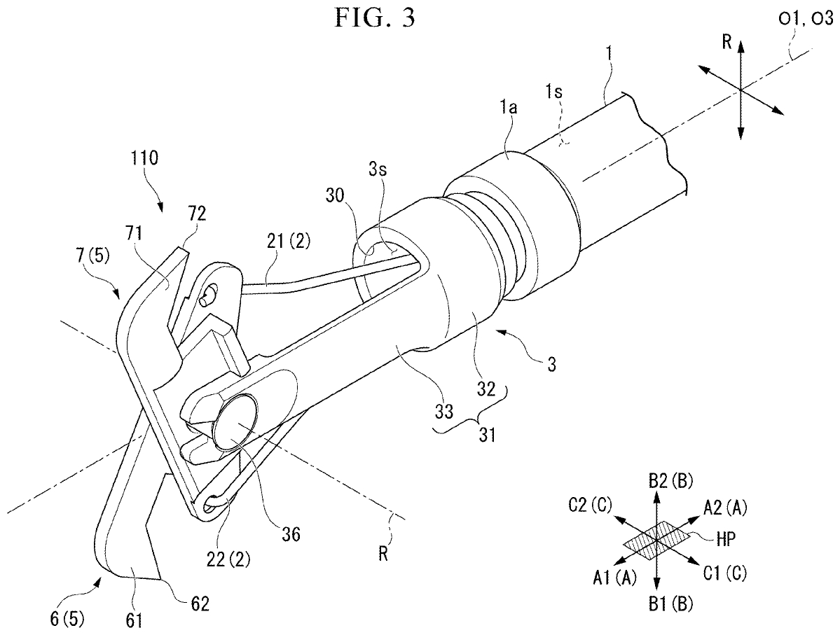

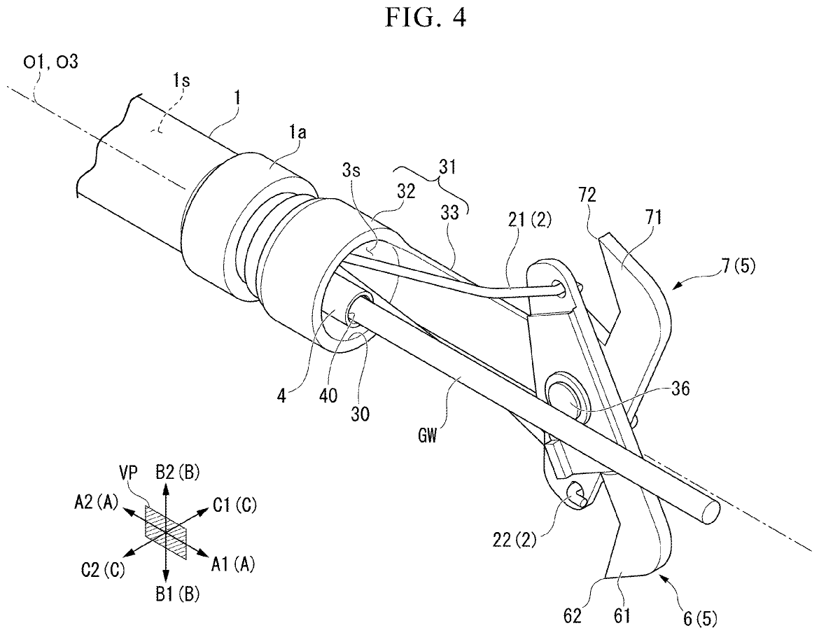

FIGS. 3 and 4 are perspective views of the distal end of the treatment tool 100.

A support member 3 and forceps jaws 5 are provided at the distal end of the treatment tool 100. The forceps jaws 5 are rotatably supported by the support member 3. The support member 3 and the forceps jaws 5 constitute a “treatment portion 110” used to treat the affected area.

In the following description, the direction in which the forceps jaws 5 open and close will be referred to as the “opening/closing direction B” or the “vertical direction B.” Further, a direction perpendicular to the longitudinal direction A and the opening/closing direction B is referred to as a “width direction C” or a “horizontal direction C.” Further, a surface horizontal to the longitudinal direction A and the width direction C is referred to as a “horizontal surface HP”. A plane horizontal to the longitudinal direction A and the opening/closing direction B is defined as a “vertical plane VP.”

The sheath 1 is a flexible and elongated member extending from a distal end 1a to a proximal end 1b. The sheath 1 has an outer diameter that allows it to be inserted into the treatment tool channel 230 of the endoscope 200. As shown in FIG. 1, when the sheath 1 is inserted into the treatment tool channel 230, the distal end 1a of the sheath 1 can protrude and retract from the distal portion 230a of the treatment tool channel 230. The sheath 1 may have insulation properties. The operating wire 2 and the guide wire tube 4 are inserted into an internal space (lumen) 1s of the sheath 1.

The operating wire 2 is a metal wire, and is inserted through the internal space 1s of the sheath 1. The distal end of the operating wire 2 is connected to the forceps jaws 5, and the proximal end of the operating wire 2 is connected to the operating portion 8.

As shown in FIG. 3, the operating wire 2 includes a first operating wire 21 and a second operating wire 22. The first operating wire 21 and the second operating wire 22 are inserted through the internal space 1s of the sheath 1.

The support member 3 is attached to the distal end 1a of the sheath 1 and supports the forceps jaws 5. The support member 3 includes a frame 31 and a pin (rotation shaft, rotation shaft member) 36. The forceps jaws 5 are supported by the pin 36 attached to the frame 31.

The frame 31 is made of metal such as stainless steel. The frame 31 includes a support main body 32 formed in a cylindrical shape and a frame piece 33 formed in a plate shape.

The support body 32 is fixed to the distal end la of the sheath 1 by caulking or the like. A central axis O3 of the support body 32 in the longitudinal direction A substantially coincides with a central axis O1 of the sheath 1 in the longitudinal direction A. An internal space 3s of the support body 32 communicates with the internal space 1s of the sheath 1.

The operating wire 2 and the guide wire tube 4 pass through the internal space 1s of the sheath 1 and the internal space 3s of the support body 32, and protrude from the distal opening 30 of the support body 32 toward the distal side A1.

The frame piece 33 is provided to protrude from the support body 32 toward the distal side A1. The frame piece 33 is formed into a flat plate shape extending in the longitudinal direction A. The thickness direction of the frame piece 33 substantially coincides with the width direction C. The proximal side A2 of the frame piece 33 is continuous with the support body 32.

The pin (rotating shaft, rotating shaft member) 36 is formed into a substantially cylindrical shape from metal such as stainless steel. The pin 36 is attached to the distal end of the frame piece 33.

The forceps jaw (first device) 5 is a member that collects living tissue. The forceps jaw 5 is made of a metal material such as stainless steel, and includes a first forceps piece 6 and a second forceps piece 7.

The first forceps piece 6 is rotatably supported by the pin 36 relative to the sheath 1 and the support member 3 with the central axis R of the pin 36 as the rotation center. The first forceps piece 6 is operated by a first operating wire 21 connected to the proximal side A2 of the first forceps piece 6.

A first edge 61 is provided on the outer peripheral surface of the first forceps piece 6 and extends outward in the radial direction R of the sheath 1 when the forceps jaws 5 are closed. A distal end 62 of the first edge 61 is formed sharply. The first forceps piece 6 is connected to the sheath 1 via the support member 3 so that the first edge 61 can move outward in the radial direction R relative to the outer circumferential surface 10 of the sheath 1.

The second forceps piece 7 is rotatably supported by the pin 36 relative to the sheath 1 and the support member 3 with the central axis R of the pin 36 as the rotation center. The second forceps piece 7 is operated by a second operating wire 22 connected to the proximal side A2 of the second forceps piece 7.

A second edge 71 is provided on the outer peripheral surface of the second forceps piece 7 and extends outward in the radial direction R of the sheath 1 when the forceps jaws 5 are closed. A distal end 72 of the second edge 71 is formed sharply. The second forceps piece 7 is connected to the sheath 1 via the support member 3 so that the second edge 71 can move outward in the radial direction R relative to the outer circumferential surface 10 of the sheath 1.

The forceps jaw 5 may further include a link mechanism and be operated by a single operating wire connected to the link mechanism.

In the following description, the direction in which the first forceps piece 6 opens is referred to as “lower side B1” in the opening/closing direction B, and the direction in which the second forceps piece 7 opens is referred to as “upper side B2” in the opening/closing direction B. Further, when looking from the distal end side A1 to the proximal side A2, the right direction in the width direction C is referred to as a “right side C1”, and the left direction is referred to as a “left side C2” in the width direction C.

The operating portion 8 is provided on the proximal side A2 of the sheath 1. The operating portion 8 includes an operating portion main body 81, a slider 83, and a guide wire port 84.

A distal end 81a of the operating portion main body 81 is connected to the proximal end 1b of the sheath 1. An operating wire 2 extends from the proximal 1b of the sheath 1. The operating wire 2 is fixed to the slider 83.

The slider 83 is attached to the operating portion main body 81 so as to be movable along the longitudinal direction A. A proximal end of the operating wire 2 is fixed to the slider 83. When the user moves the slider 83 forward and backward relative to the operating portion main body 81, the operating wire 2 moves forward and backward.

The guide wire port 84 is an opening into which the guide wire GW can be inserted into the guide wire tube 4. As shown in FIG. 4, the guidewire GW inserted from the guidewire port 84 passes through the guidewire tube 4 and projects from the distal end opening 40 of the guidewire tube 4 toward the distal end side A1.

### Operation of Endoscopic Treatment System 300

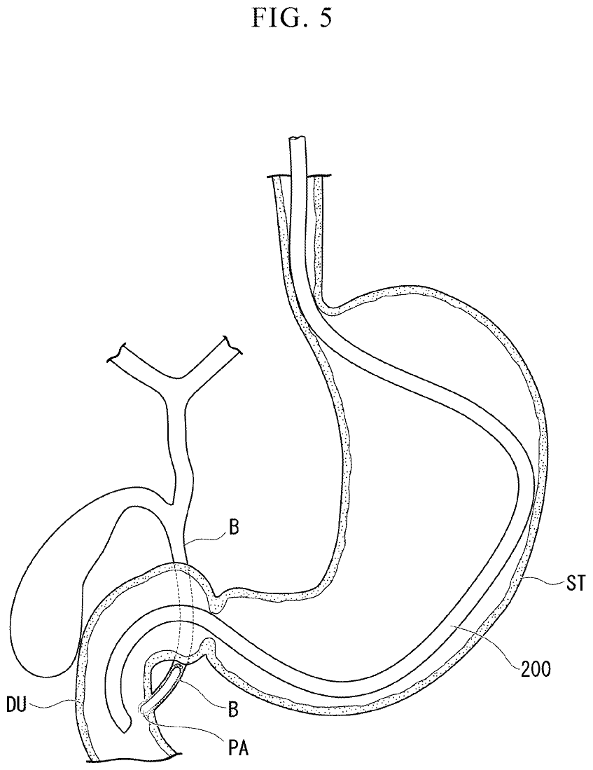

Next, the operation of the endoscopic treatment system 300 according to this embodiment will be described. Specifically, a procedure for placing a stent 430 in the bile duct B using endoscopic retrograde cholangiopancreatography (ERCP) will be described. FIG. 5 is a diagram showing the bile duct B to be treated.

### Step S1: Endoscope Insertion Step

The operator inserts the insertion portion 210 of the endoscope 200 into the patient's lumen through a natural opening such as the mouth. The operator operates the operating portion 220 to bend the bending portion 212 as necessary. The operator inserts the rigid distal portion 211 of the endoscope 200 up to the duodenum DU. At this time, the operator positions the rigid distal portion 211 at a position that captures the opening PA1 of the papilla PA in front, so that the rigid distal portion 211 is aligned so that the protruding direction (or protruding axis) of the treatment tool 100 and the bile duct direction (or bile duct axis) match. The operator may insert the insertion portion 210 of the endoscope 200 into the patient's lumen using an overtube.

### Step S2: First Device Insertion Step

The operator inserts the treatment tool 100 into the bile duct B from the opening PA1 of the papilla PA. Specifically, the treatment tool 100 is inserted into the treatment tool channel 230 of the endoscope 200, and the forceps jaw (first device) 5 of the treatment tool 100 is made to protrude from the distal portion 230a. The forceps jaw (first device) 5 of the treatment tool 100 is inserted into the bile duct B from the opening PA1 of the papilla PA while keeping the device closed.

### Step S3: Hooking Step

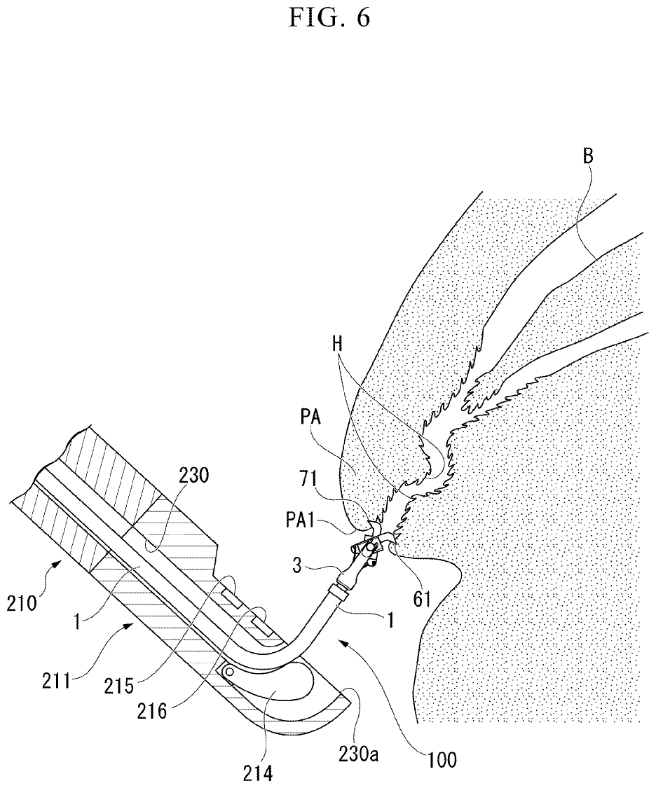

FIG. 6 is a diagram showing the hooking step.

The operator opens the forceps jaws 5 and hooks the edges of the forceps jaws 5 (first edge 61 and second edge 71) to a lumen wall of hollow organ in the papilla PA in a region closer to the opening PA1 of the papilla PA than a part where the fold H in the bile duct B is located.

### Step S4: Second Device Insertion Step

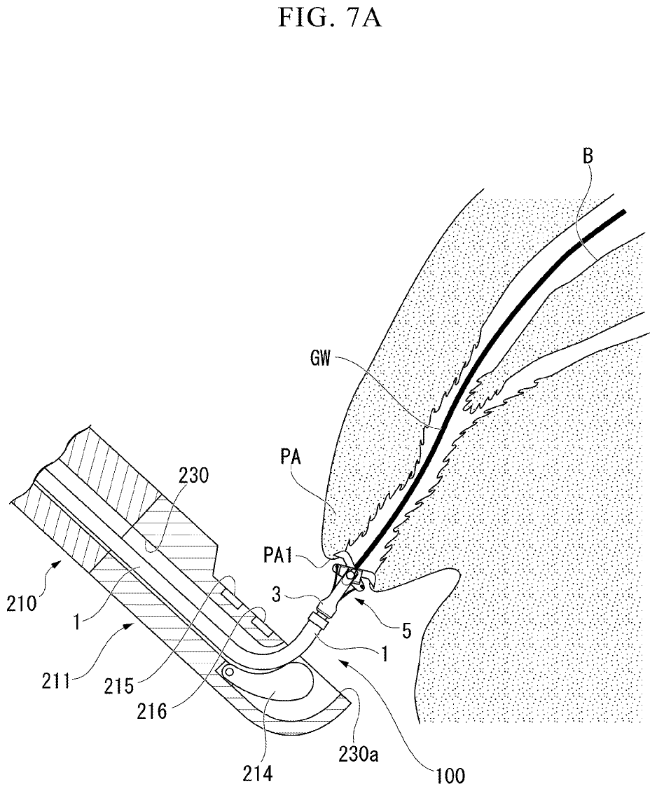

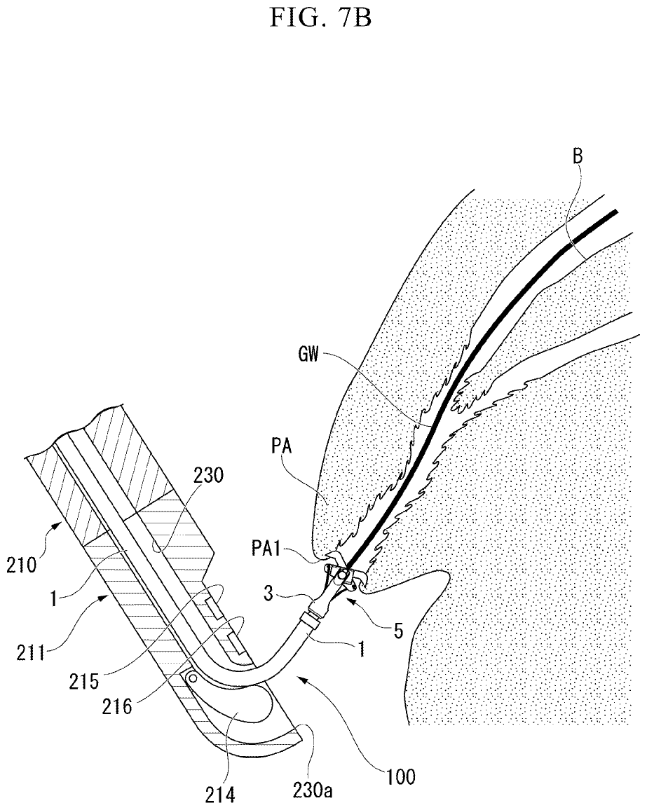

FIGS. 7A and 7B are diagrams showing the second device insertion step.

In the second device insertion step, the operator advances the guide wire (second device) GW within the bile duct B while pulling the papilla PA with the forceps jaws (first device) 5, and inserts the guide wire (second device) GW into bile duct B. The operator may pull the papilla PA by retracting the forceps jaws (first device) 5, as shown in FIG. 7A. Further, as shown in FIG. 7B, the operator may pull the papilla PA by curving the bending portion 212 (angle operation) so that the rigid distal portion 211 of the endoscope 200 moves away from the papilla PA. In addition, in the second device insertion step, it is not necessarily necessary to advance the guide wire (second device) GW in the bile duct B at the same time as the operation of pulling the papilla PA with the forceps jaw (first device) 5, and the guide wire (second device) GW may be inserted into the bile duct B while the papilla PA is pulled with the forceps jaws (first device) 5.

The shape of the bile duct B in the papilla PA may be such that it is difficult to insert the guide wire GW. For example, this may occur if the bile duct B is sharply bent or meandering in the papilla PA, or if part of the bile duct B is narrowed due to folds H or the like. In addition, the fold H and the bile duct near the opening PA1 are very soft, and the bile duct may be easily bent or buckled just by lightly pushing the treatment tool 100 around the opening PA1. In particular, when the bile duct B has a NDS (Narrow Distal Segment; an area where the duodenal penetration part distal to the extrabiliary bile duct is narrowed due to the influence of the sphincter of Oddi muscle), it becomes difficult to insert the guide wire GW into the bile duct B. According to the second device insertion step described above, by stretching the papilla PA by pulling the papilla PA with the forceps jaws 5, the shape of the bile duct B can be made into a shape into which the guide wire GW can be easily inserted. It is desirable for the operator to pull the papilla PA with the forceps jaws 5 until the bile duct B has a straight shape. The operator does not necessarily have to pull the papilla PA until the shape of the bile duct B becomes straight. The operator only has to pull the papilla PA until the shape of the bile duct B changes into a shape that allows easy insertion of the guide wire GW. For example, the operator may pull the papilla PA until the shape of the bile duct B changes into a gently curved shape.

### Step S5: Guide Wire Placement Step

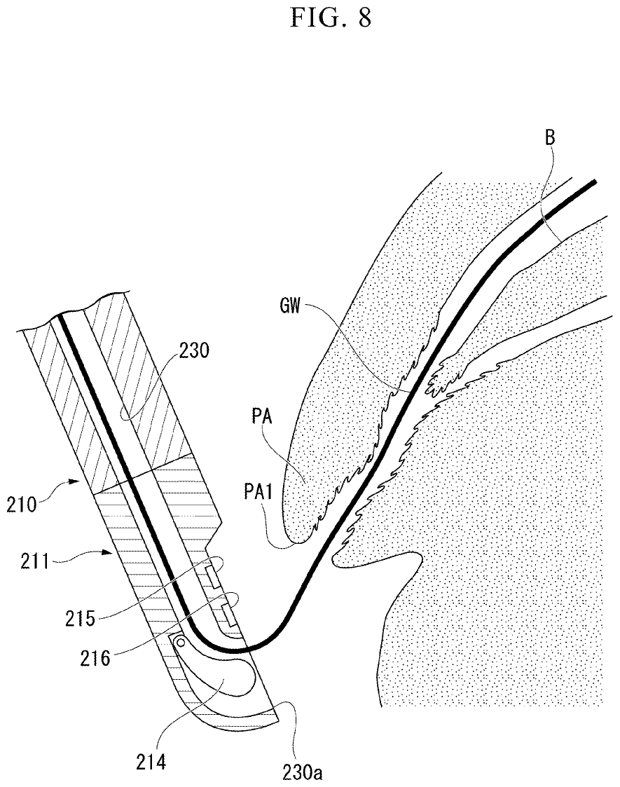

FIG. 8 is a diagram showing the guide wire placement step.

The operator withdraws the treatment tool 100 while leaving the guide wire GW indwelling in the bile duct B. Thereby, only the guide wire GW is placed in the bile duct B.

### Step S6: Stent Placement Step

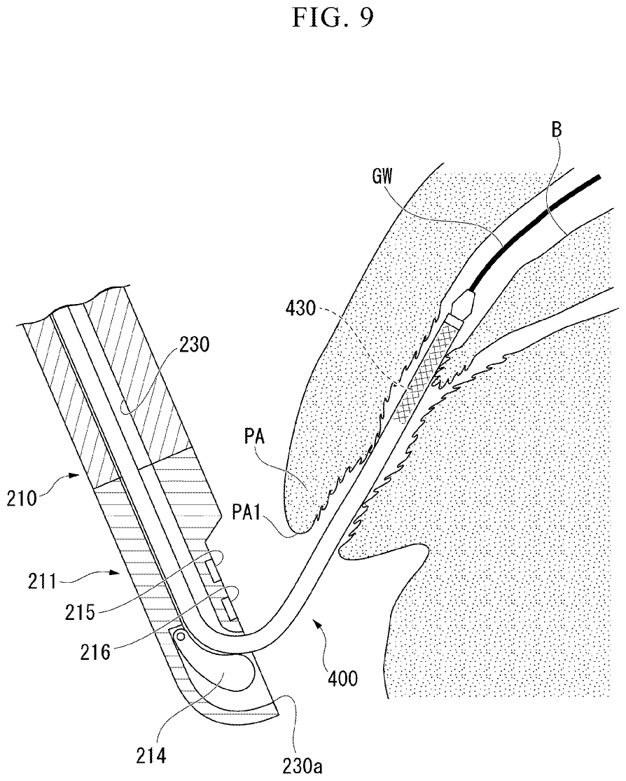

FIG. 9 is a diagram showing the stent placement step.

The operator inserts a stent delivery device 400 into the bile duct B along the guide wire GW. The operator operates the stent delivery device 400 to move the stent delivery device 400 toward the liver along the guide wire GW, and moves the portion of the stent delivery device 400 in which the stent 430 is accommodated to the vicinity of the stenosis. The operator operates the stent delivery device 400 to expose the accommodated stent 430 and indwells the stent 430.

Although the procedure for indwelling the stent 430 has been shown as an example of the operation of the endoscopic treatment system 300, treatments using the endoscopic treatment system 300 are not limited to the procedure for indwelling the stent 430. For example, the endoscopic treatment system 300 can also be used for procedures using a stone quarrying/lithotomy basket or a stone quarrying balloon using an indwelling guidewire GW.

According to the endoscopic treatment tool 100 and the method for insertion into the bile duct B according to the present embodiment, it is easy to insert the guide wire (second device) GW into the bile duct. Furthermore, it is easy to insert a medical device such as the stent delivery device 400 into the bile duct using the guide wire (second device) GW inserted into the bile duct.

Although the first embodiment of the present invention has been described above in detail with reference to the drawings, the specific configuration is not limited to this embodiment, and design changes may be made within the scope of the gist of the present invention. Moreover, the components shown in the above-described embodiments and modified examples can be configured by appropriately combining them.

### Modification 1-1

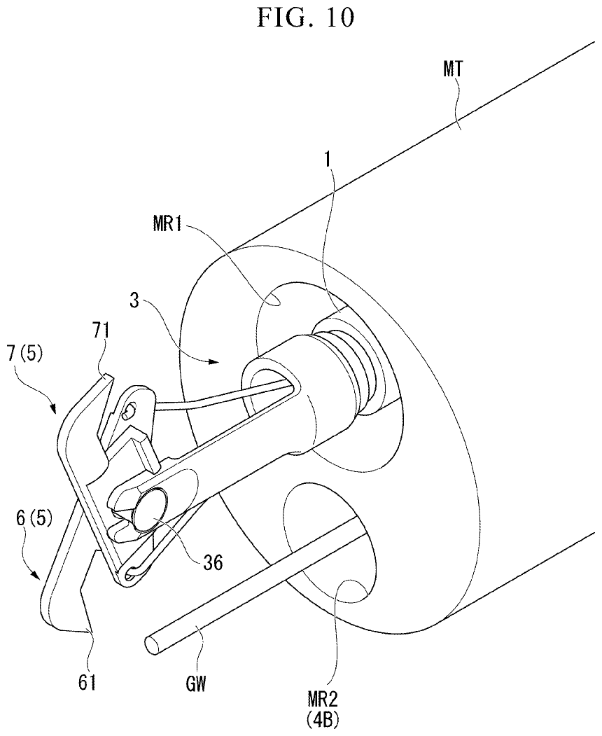

In the above embodiment, the guide wire tube 4, through which the guide wire (second device) GW is inserted, is inserted through the sheath 1. However, the tube through which the guide wire (second device) GW is inserted is not limited to this. FIG. 10 is a diagram showing a guidewire lumen 4B that is a modified example of the guidewire tube 4. The guide wire lumen 4B may be one of a plurality of lumens formed in the multi-lumen tube MT. As shown in FIG. 10, the treatment tool 100 (not including the guide wire tube 4) may be inserted into the first lumen MR1 of the multi-lumen tube MT, and the guide wire GW may be inserted into the second lumen MR2.

### Modification 1-2

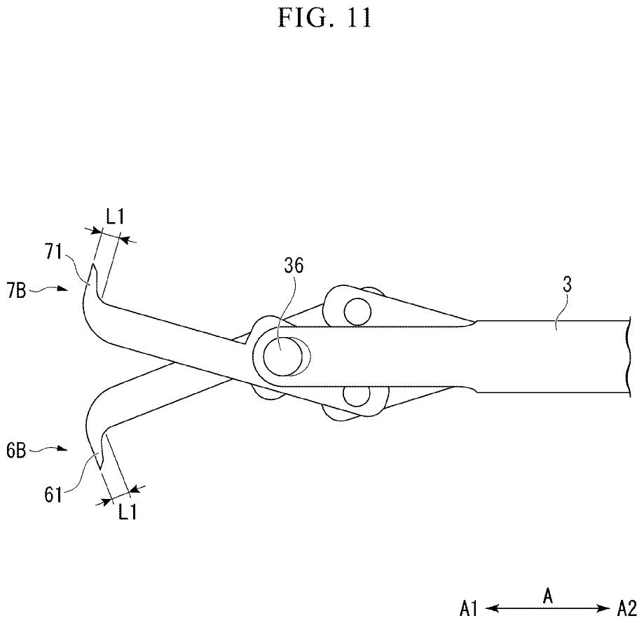

FIG. 11 is a diagram showing a forceps jaw 5B that is a modification of the forceps jaw 5.

The forceps jaw 5B includes a first forceps piece 6B and a second forceps piece 7B. A first edge 61B of the first forceps piece 6B and a second edge 71B of the second forceps piece 7B have a shorter length L1 in the longitudinal direction A compared to the first edge 61 and second edge 71 of the first embodiment.

### Modification 1-3

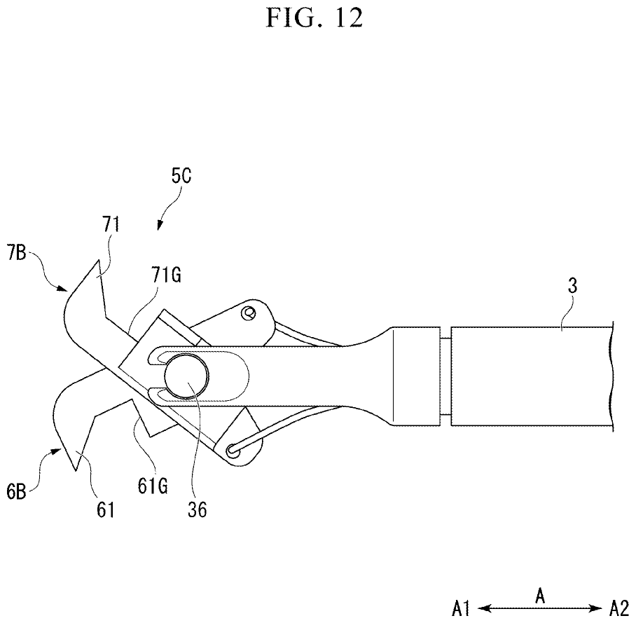

FIG. 12 is a diagram showing a forceps jaw 5C that is a modification of the forceps jaw 5.

The forceps jaw 5C includes a first forceps piece 6C and a second forceps piece 7C. The first forceps piece 6C has a first edge 61 and a first groove 61G on the proximal side of the first edge 61. The second forceps piece 7C has a second edge 71 and a second groove 71G on the proximal side A2 of the second edge 71.

### Modification 1-4

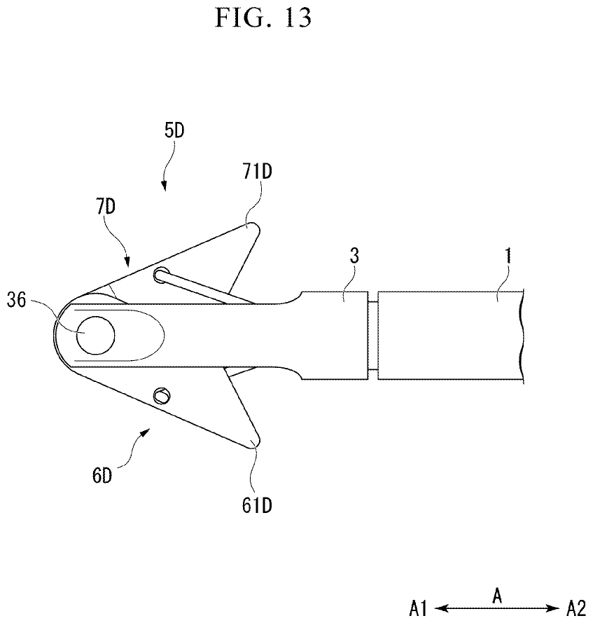

FIG. 13 is a diagram showing a forceps jaw 5D that is a modification of the forceps jaw 5.

The proximal end A1 of the forceps jaws 5D opens and closes. The forceps jaw 5D includes a first forceps piece 6D and a second forceps piece 7D. A first edge 61D extending outward in the radial direction R of the sheath 1 is provided on the outer peripheral surface of the first forceps piece 6D. A second edge 71D extending outward in the radial direction R of the sheath 1 is provided on the outer peripheral surface of the second forceps piece 7D.

### Modification 1-5

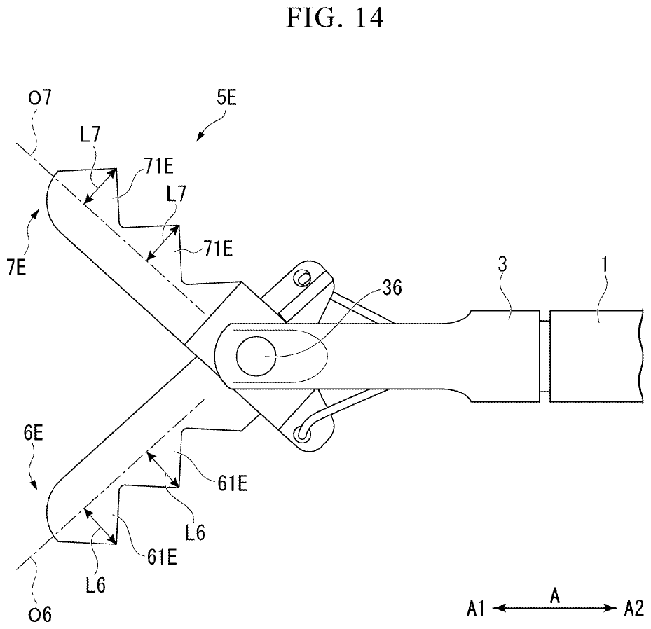

FIG. 14 is a diagram showing a forceps jaw 5E that is a modification of the forceps jaw 5.

The forceps jaw 5E includes a first forceps piece 6E and a second forceps piece 7E.

The outer peripheral surface of the first forceps piece 6E is provided with a plurality of first edges 61E that extend outward in the radial direction R of the sheath 1 when the forceps jaws 5E are closed. The heights of the plurality of first edges 61E (the length from a central axis O6 in the longitudinal direction A of the first forceps piece 6E) are the same.

The outer peripheral surface of the second forceps piece 7E is provided with a plurality of second edges 71E that extend outward in the radial direction R of the sheath 1 when the forceps jaws 5E are closed. The heights (lengths from a central axis O7 in the longitudinal direction A of the second forceps piece 7E) of the plurality of second edges 71E are the same.

### Modification 1-6

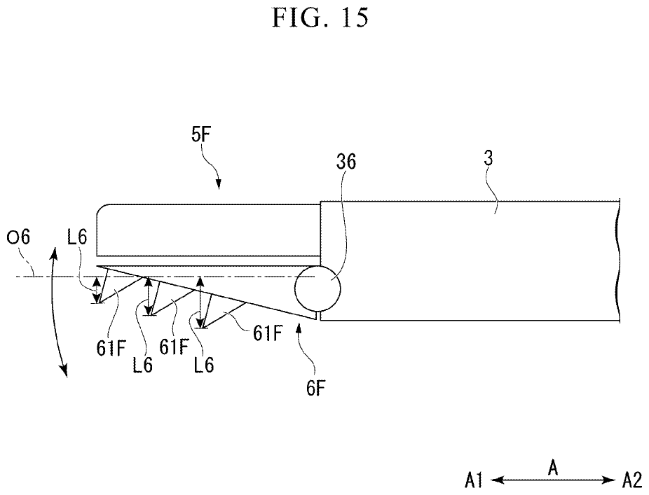

FIG. 15 is a diagram showing a forceps jaw 5F, which is a modification of the forceps jaw 5.

The forceps jaw 5F has a first forceps piece 6F and a fixed forceps piece 9F. The fixed forceps piece 9F does not move. The forceps jaw 5F is a single-open forceps jaw.

The outer peripheral surface of the first forceps piece 6F is provided with a plurality of first edges 61F that extend outward in the radial direction R of the sheath 1 when the forceps jaws 5F are closed. The heights (lengths from the central axis O6 in the longitudinal direction A of the first forceps piece 6F) of the plurality of first edges 61F are different.

### Second Embodiment

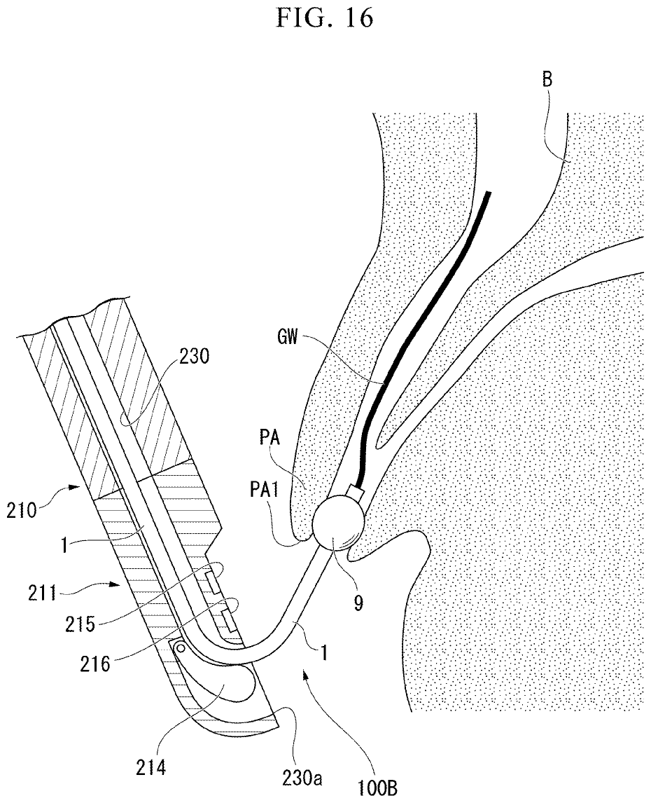

An endoscopic treatment tool 100B according to a second embodiment of the present invention will be described with reference to FIG. 16. In the following description, components that are common to those already described will be given the same reference numerals and redundant description will be omitted.

### Endoscope Treatment Tool 100B

FIG. 16 is a diagram showing the second device insertion step using the endoscopic treatment tool 100B.

The endoscopic treatment tool 100B (also referred to as the treatment tool 100B) is used as an endoscopic treatment system together with the endoscope 200, similarly to the endoscopic treatment tool 100 of the first embodiment. The treatment tool 100B includes a sheath 1, an operation wire 2, a support member 3, a balloon (first device) 9, and an operating portion 8. The treatment tool 100B differs from the endoscopic treatment tool 100 according to the first embodiment in that it includes a balloon 9 instead of the forceps jaws 5.

The balloon (first device) 9 is provided at the distal end of the sheath 1 and can be expanded by fluid (gas or liquid) supplied from the operating portion 8.

As shown in FIG. 16, in the second device insertion step, the operator advances the guide wire (second device) GW within the bile duct B while pulling the papilla PA with the expanded balloon (first device) 9, so that the guide wire (second device) GW is inserted into the bile duct B.

According to the endoscopic treatment tool 100B and the method for insertion into the bile duct B according to the present embodiment, it is easy to insert the guide wire (second device) GW into the bile duct. Furthermore, it is easy to insert a medical device such as the stent delivery device 400 into the bile duct using the guide wire (second device) GW inserted into the bile duct.

Although the second embodiment of the present invention has been described above in detail with reference to the drawings, the specific configuration is not limited to this embodiment, and design changes may be made within the scope of the gist of the present invention. Moreover, the components shown in the above-described embodiments and modified examples can be configured by appropriately combining them.

What is claimed is:

1. A method for insertion into a bile duct, comprising:
inserting a first device from a duodenum into an opening of a papilla; and
advancing a second device within the bile duct while the papilla is pulled by the first device.

2. The insertion method according to claim 1, further comprising:
hooking the first device onto a lumen wall of hollow organ within the papilla, before advancing the second device within the bile duct while the papilla is pulled by the first device.

3. The insertion method according to claim 1, further comprising:
removing the first device from an endoscope while a distal end of the second device indwells within the bile duct, after advancing the second device within the bile duct while the papilla is pulled by the first device.

4. The insertion method according to claim 1, wherein
the second device is a guide wire,
the first device has a lumen through which the guide wire can be inserted, and
the guide wire is advanced within the lumen while the papilla is pulled by the first device.

5. The insertion method according to claim 1, wherein
the second device is a guide wire,
the first device is inserted into a first lumen of a multi-lumen tube, and
the guide wire is advanced within a second lumen of the multi-lumen tube while the papilla is pulled by the first device.

6. A method for insertion into the bile duct, comprising:
inserting a first device from a duodenum into an opening of a papilla; and
advancing a second device within the bile duct while pulling the papilla with the first device.

7. The insertion method according to claim 6, further comprising:
hooking the first device onto a lumen wall of hollow organ within the papilla, before advancing the second device within the bile duct while pulling the papilla with the first device.

8. The insertion method according to claim 6, further comprising:
hooking the first device to a region closer to the opening of the papilla than to a part of the bile duct where there is a fold.

9. The insertion method according to claim 6, further comprising:
removing the first device from an endoscope while a distal end of the second device indwells in the bile duct, after advancing the second device within the bile duct while pulling the papilla with the first device.

10. The insertion method according to claim 6, wherein
the second device is a guide wire.

11. The insertion method according to claim 6, wherein
the first device is a forceps jaw having an edge on its outer peripheral surface.

12. The insertion method according to claim 6, wherein
the first device is a balloon.

13. The insertion method according to claim 10, wherein
the first device has a lumen through which the guide wire can be inserted, and
the guide wire is advanced within the lumen while pulling the papilla with the first device.

14. The insertion method according to claim 10, wherein
the first device is inserted into a first lumen of a multi-lumen tube, and
the guide wire is advanced within a second lumen of the multi-lumen tube while pulling the papilla with the first device.

15. An endoscopic treatment tool, comprising:
a sheath; and
a forceps jaw movable relative to the sheath,
wherein an outer circumferential surface of the forceps jaw has at least one edge facing outward in a radial direction of the sheath, and
the forceps jaw is connected to the sheath so that the edge can be moved in the radial direction outward relative to the outer peripheral surface of the sheath.

16. The endoscopic treatment tool according to claim 15, further comprising:
a tube into which a guide wire can be inserted,
wherein the tube is inserted through the sheath.

17. The endoscopic treatment tool according to claim 15, further comprising:
a rotation shaft that rotatably supports the forceps jaw with respect to the sheath.

18. The endoscopic treatment tool according to claim 15, wherein
the sheath has a lumen into which a guide wire can be inserted.

19. The endoscopic treatment tool according to claim 15, wherein
the at least one edge is a plurality of edges, and
heights of the plurality of edges are different.

20. The endoscopic treatment tool according to claim 15, wherein
the at least one edge is a plurality of edges, and
heights of the plurality of edges are the same.
